(19) United States
(12) Patent Application Publication
(10) Pub. No.: US 2009/0112226 A1
(43) Pub. Date: Apr. 30, 2009

(54) POLYP TOME

(75) Inventors: Thomas G. Self, Durham, NC (US); David F. Waller, Tampa, FL (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/258,021

(22) Filed: Oct. 24, 2008

Related U.S. Application Data
(60) Provisional application No. 60/983,673, filed on Oct. 30, 2007.

(51) Int. Cl. A61B 17/26

(52) U.S. Cl. 606/113

(57) ABSTRACT

The present invention provides apparatus and methods for facilitating removal of polyps from a bodily cavity, such as the colon. The apparatus comprises a catheter having proximal and distal regions, and a cutting wire extending through a first lumen of the catheter. At least one notched section is formed in the distal region of the catheter, and at least a portion of the cutting wire is visibly exposed at the notched section. At the notched section, the exposed cutting wire does not extend substantially radially outward beyond a main body of the catheter. Therefore, the profile of the catheter is not increased. A stiffening member may be advanced within a second lumen of the catheter to selectively provide support to the cutting wire in the vicinity of the notched section. If the stiffening member is not employed, the cutting wire may be proximally retracted or tensioned to bend the distal region of the catheter in the vicinity of the notched section.

### PRIORITY CLAIM

This invention claims the benefit of priority of U.S. Provisional Application Ser. No. 60/983,673, entitled “Polyp Tome,” filed Oct. 30, 2007, the disclosure of which is hereby incorporated by reference in its entirety.

### BACKGROUND

The present invention relates to medical devices, and more particularly, to apparatus and methods for surgically treating polyps.

In the United States, cancer is the second leading cause of death with more than 500,000 fatalities per year. Colorectal cancer accounts for about 5-20% of the total number of annual cancer deaths. While the majority of people with colorectal cancer are 50 years of age or older, the risk may increase around age 40, and men are more susceptible than women of developing colorectal cancer.

While cancers of the colon and rectum are often fatal, a significant number are highly preventable if treated at an early stage. Most of these cancers begin as an adenomatous polyp, which is a growth of tissue that protrudes from the lining of the colon or rectum into the space inside the bowel. As many as 20 percent of adults over 40 years old may have one or more colorectal polyps.

Some colorectal polyps may be accompanied with symptoms such as abdominal pain, rectal bleeding, and abnormal bowel habits such as constipation and diarrhea. However, many other polyps are not accompanied by symptoms, and therefore, it is highly recommended to have regular screenings regardless of whether symptoms are present. Most colorectal polyps that are found in the early stages can be removed safely and completely. However, if left untreated, some polyps may turn cancerous over time. In general, the larger a polyp, the greater the likelihood of cancer.

A colonoscopy or sigmoidoscopy may be performed to detect the existence of polyps. A doctor is likely to remove all polyps discovered during a bowel examination, and once removed, a pathologist may examine the polyps under a microscope to determine whether the polyps are cancerous. Once the section of colon that contains the polyp is removed, there is a chance of subsequently developing new polyps in other areas of the colon, and therefore, follow-up examinations are very important.

One present technique for removing polyps is to use a looped snare. In this technique, a wire loop cuts the base of the polyp and cauterizes it to prevent bleeding. This requires looping the snare over the head of the polyp, then positioning the loop over the base prior to cauterization. One drawback associated with this technique is that some polyps may be too large to be snared, i.e., it may be difficult or impractical to position the loop of the snare over the head of a large polyp. If the polyp is too large to snare, it may need to be surgically removed using laparoscopic or open surgical techniques.

In view of the foregoing, there is a need for a device that facilitates treatment of polyps of all shapes and sizes quickly and effectively, without the need for laparoscopic or open surgery.

### SUMMARY

The present invention provides apparatus and methods for facilitating removal of polyps from a bodily cavity, such as the colon or rectum. In a first embodiment, the apparatus comprises a catheter having proximal and distal regions, and further having a first lumen extending between the proximal and distal regions. A cutting wire having proximal and distal ends extends through the first lumen of the catheter. At least one notched section is formed in the distal region of the catheter, wherein at least a portion of the cutting wire is visibly exposed at the notched section. At the notched section, the exposed cutting wire does not extend substantially radially outward beyond a main body of the catheter. Therefore, the profile of the catheter is not increased by the exposed cutting wire.

A stiffening member having proximal and distal ends may be distally advanced within a second lumen of the catheter to selectively provide support to the cutting wire in the vicinity of the notched section. Alternatively, the stiffening member may be rigidly affixed adjacent to the notched section to provide support to the cutting wire.

The distal end of the cutting wire is affixed to an affixation member positioned near a distal end of the catheter. If the stiffening member is not employed, or if the stiffening member is positioned proximal to the notched section, the cutting wire may be proximally retracted or tensioned to bend the distal region of the catheter in the vicinity of the notched section.

In an alternative embodiment, an indented surface of the notched section may comprise a curvature, such as a concave shape, formed between first and second ends of the notched section. The curvature of the indented surface may vary the distance between the catheter shaft and the cutting wire along the notched section.

In use, the catheter may be delivered through a working channel of an endoscope that has been positioned in the bodily cavity. An electrical current is provided to the cutting wire to perform an electrosurgical procedure to remove the polyp. The stiffening member may be positioned adjacent to the notched section to provide support to the cutting wire during removal of the polyp.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side-sectional view of a distal region of an apparatus for treating a polyp.

FIG. 2A illustrates a cross-sectional view along line A-A of FIG. 1.

FIG. 2B illustrates a cross-sectional view along line B-B of FIG. 1.

FIG. 3 is a side view of a proximal handle portion that may be used in conjunction with the apparatus of FIG. 1.

FIG. 4 is a side view of a distal region of an alternative embodiment of the apparatus of FIG. 1.

FIG. 5 is a side view of the apparatus of FIG. 4 in a curved configuration.

FIGS. 6A-6B are, respectively, perspective views of the distal ends of exemplary side-viewing and end-viewing endoscopes that may be used in conjunction with the systems of FIGS. 1-2 and FIGS. 4-5.

FIG. 7 is a perspective view of an exemplary method of treating a polyp using the apparatus of FIG. 1.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patent's anatomy during a medical procedure.

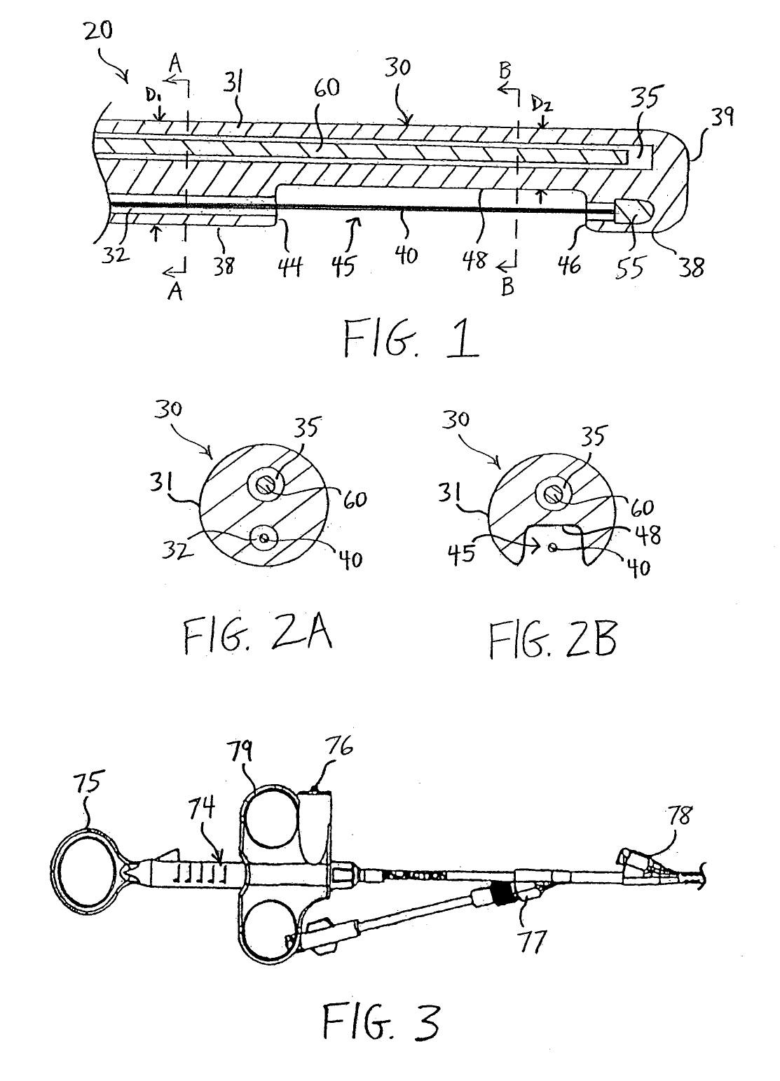

The present embodiments relate to apparatus and methods for surgically treating polyps. Referring now to FIG. 1, in a first embodiment, apparatus 20 comprises a catheter 30 having proximal and distal regions, and further having a first lumen 32 extending between the proximal and distal regions. A cutting wire 40, which has proximal and distal ends, extends through the first lumen 32 of the catheter 30.

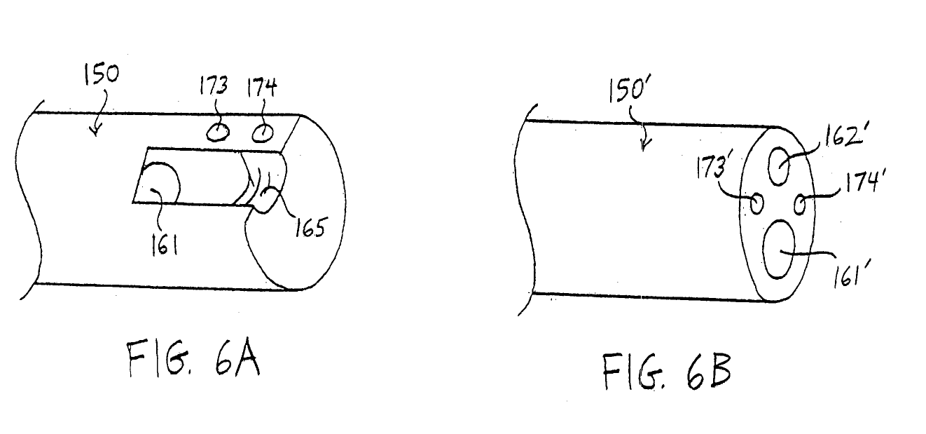

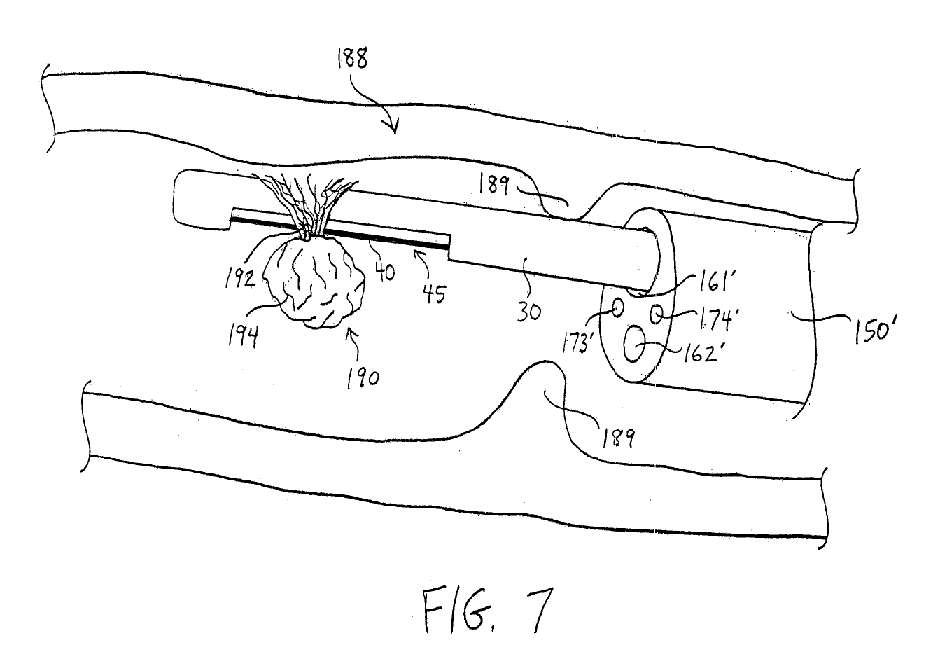

The catheter 30 preferably comprises a flexible, tubular member that may be formed from one or more semi-rigid polymers. For example, the catheter 30 may be manufactured from polyurethane, polyethylene, tetrafluoroethylene, polytetrafluoroethylene, perfluoalkoxl, fluorinated ethylene propylene, or the like. The catheter may have a length and an outer diameter sufficient to extend through a working channel of conventional endoscope (see FIGS. 6-7, below). In the embodiment illustrated, the catheter 30 has an approximate overall length of about 230 cm. Further exemplary dimensions are noted below.

The catheter 30 also may comprise a hydrophilic coating (not shown) overlying its outer surface. The hydrophilic coating, when applied to the outer surface of the catheter 30, imparts suppleness and kink resistance to the catheter. The hydrophilic coating also may provide a lubricated surface to facilitate movement through the working channel of the endoscope.

The cutting wire 40 may comprise any suitable wire configuration. For example, the cutting wire 40 may comprise a monofilament cutting wire that may provide for more concentrated transmission to the target tissue, as explained further below. Alternatively, a braided cutting wire may be employed and may be useful for facilitating coagulation of the cut tissue. Still further, any suitable configuration of cutting wire may be employed, and may be tailored for a particular procedure. The cutting wire 40 may comprise a thickness suitable for cutting a polyp, and in one embodiment, may comprise a thickness in the range of about 0.2 to 1.0 mm.

In accordance with one aspect, at least one notched section 45 is formed in the distal region of the catheter 30, and at least a portion of the cutting wire 40 is exposed in the notched section 45, as shown in FIG. 1. The notched section 45 is formed between a first end 44 and a second end 46, which define the proximal and distal ends of the notched section 45, respectively.

At a location proximal to the first end 44 of the notched section 45, the catheter 30 has a main body 31 having an outer region 38 defining a first outer diameter D₁ of the catheter. Similarly, at a location distal to the second end 46 of the notched section 45, the main body 31 preferably comprises an outer region 38 having the same first outer diameter D₁, as shown in FIG. 1. Within the notched section 45, the catheter 30 has an indented surface 48 defining a second outer diameter D₂ of the catheter. As shown in FIG. 1, the first outer diameters D₁ of the outer regions 38, which flank the notched section 45, are greater than the second outer diameter D₂ of the catheter 30.

Further, the second outer diameter D₂ may be about 40-90% of the diameter of the first outer diameter D₁. In the embodiment depicted in FIG. 1, the second outer diameter D₂ is about 70% of the diameter of the first outer diameter D₁. Solely by way of example, the first outer diameter D₁ may range from about 2.0 mm to about 6.0 mm, and more preferably about 4.0 mm. The second outer diameter D₂ may range from about 1.4 mm to about 4.2 mm, and more preferably about 2.8 mm. Further, the first lumen 32 of the catheter 30 may comprise a diameter in a range from about 0.5 mm to about 1.3 mm, and more preferably about 0.9 mm, while a second lumen 35 of the catheter 30 may comprise a diameter in a range from about 0.7 mm to about 1.7 mm, and more preferably about 1.2 mm.

It will be appreciated that the above-referenced dimensions are provided solely for exemplary purposes and are not intended to be limiting. Further, while the indented surface 48 is shown as having a substantially straight, laterally-extending configuration in FIG. 2B, the indented surface 48 may comprise a concave laterally-extending configuration, whereby a central portion of the indented surface 48 is spaced further from the cutting wire 40.

The notched section 45 may comprise any suitable configuration and dimensions for performing the functions described herein. For example, a longitudinal length of the notched section 45, i.e., the distance between the first end 44 and the second end 46, may range from a few millimeters to several centimeters. Preferably, the longitudinal length of the notched section 45 is greater than at least the base portion 192 of a polyp 190 (see FIG. 7). For example, if the base portion 192 of the polyp 190 is relatively small, e.g., 0.5 mm to about 4.0 mm, then the longitudinal length of the notched section 45 may range from about 2.0 mm to about 15.0 mm. If the base portion 192 of the polyp 190 is relatively large, e.g., 5.0 mm to about 12.0 mm, then the longitudinal length of the notched section 45 may range from about 20 mm to about 60 mm, or even greater if desired. Accordingly, depending on the size and configuration of a polyp, the longitudinal length of the notched section 45 may range from about 2.0 mm to about 60 mm or more in order to accommodate at least the base portion 192 of the polyp 190.

The first lumen 32 extends along a longitudinal axis of the catheter 30 at a location laterally offset from the outer region 38, and further laterally offset from the indented surface 48, as shown in FIG. 1. In this manner, the cutting wire 40 extends laterally between longitudinal axes formed by the indented surface 48 and the outer regions 38. Therefore, the exposed portion of the cutting wire 40 is spaced apart from the indented surface 48, as shown in FIG. 1. Further, the exposed portion of the cutting wire 40 does not extend substantially radially outward beyond the outer regions 38 of the main body 31. Preferably, the cutting wire 40 is tensioned so that it comprises a tautness, as shown in FIG. 1. Accordingly, the profile of the catheter 30 is not increased by the exposed portion of the cutting wire 40.

The distal end of the cutting wire 40 may be rigidly attached to an affixation member 55, which is positioned near the distal end 39 of the catheter 30. The affixation member 55 may be wedged into the catheter 30, adhered within an interior portion of the catheter, or otherwise secured to the catheter, and may comprise an anchoring feature that rigidly attaches to the cutting wire 40 to maintain the tautness of the cutting wire 40.

Preferably, the apparatus 20 further comprises a stiffening member 60, which has proximal and distal ends, and extends through the second lumen 35 of the catheter 30, as shown in FIG. 1. The stiffening member 60 may comprise any suitable material, such as stainless steel or nitinol. In one embodiment, the second lumen 35 comprises an inner diameter that is larger than an outer diameter of the stiffening member 60 to permit longitudinal advancement and retraction of the stiffening member 60 within the lumen. In this embodiment, the stiffening member 60 is adapted to be advanced to provide support to the cutting wire 40, as shown in FIG. 1, but further may be retracted proximal to the notched section 45 to allow a distal portion of the catheter 30 to bend, as explained further with respect to FIG. 5 below.

In an alternative embodiment, the stiffening member 60 may be permanently affixed adjacent to the notched section 45, such that the stiffening member 60 provides support to the cutting wire 40, but cannot be proximally retracted to permit bending of the notched section 45. In this alternative embodiment, stiffening member may span a longitudinal length equal to or slightly greater than the notched section 45, but need not span the entire longitudinal length of the catheter 30.

Referring to FIG. 3, a handle portion that may be used in conjunction with the catheter 30 is described. In use, the proximal end of the cutting wire 40 is operably coupled to a handle 74 having a thumb ring 75 and a slidable actuator 79, such that manipulation of the handle 74 causes the cutting wire 40 to move relative to the catheter 30. In the particular illustration, the actuator 79 is operably coupled with the cutting wire 40 and the thumb ring 75 is operably coupled to the catheter. Proximal retraction of the actuator 79 with respect to the thumb ring 75 causes retraction of the cutting wire 40 with respect to the catheter 30, which in turn causes the notched section 45 of the catheter shaft to be bowed with respect to a remaining portion of the catheter shaft, as shown and explained in greater detail in FIG. 5 below. By contrast, distal advancement of the actuator 79 with respect to the thumb ring 75 allows the cutting wire 40 and the notched section 45 to straighten, thereby helping the catheter 30 achieve a substantially straightened configuration along a longitudinal axis of the catheter shaft.

The handle portion of the apparatus 20 further comprises a connector 76, which electrically couples a portion of the cutting wire 40 to an electrical source (not shown) for supplying electricity to the cutting wire 40 for performing an electro surgical procedure. Further, an injection port 77 is disposed along the proximal portion of the shaft of the catheter 30, as shown in FIG. 3. The injection port 77 may provide access to a lumen adapted for the injection of fluid, such as contrast. In one embodiment, the injection port 77 may be in fluid communication with a separate lumen, such that the catheter 30 comprises at least three lumens. In an alternative embodiment, the injection port 77 may be in communication with the first lumen 32, such that fluids are injected through the cutting wire lumen and exit in the vicinity of the notched section 45.

The handle portion of the apparatus 20 further comprises a stiffening member port 78, which also is disposed along a proximal portion of the shaft of the catheter 30 and provides access to the second lumen 35 (see FIGS. 1-2). In the embodiment in which the stiffening member 60 is longitudinally movable within the second lumen 35, a proximal portion of the stiffening member 60 may be disposed outside of the port 78 for manual manipulation by a physician to effect proximal retraction and distal advancement of the stiffening member with respect to the catheter 30.

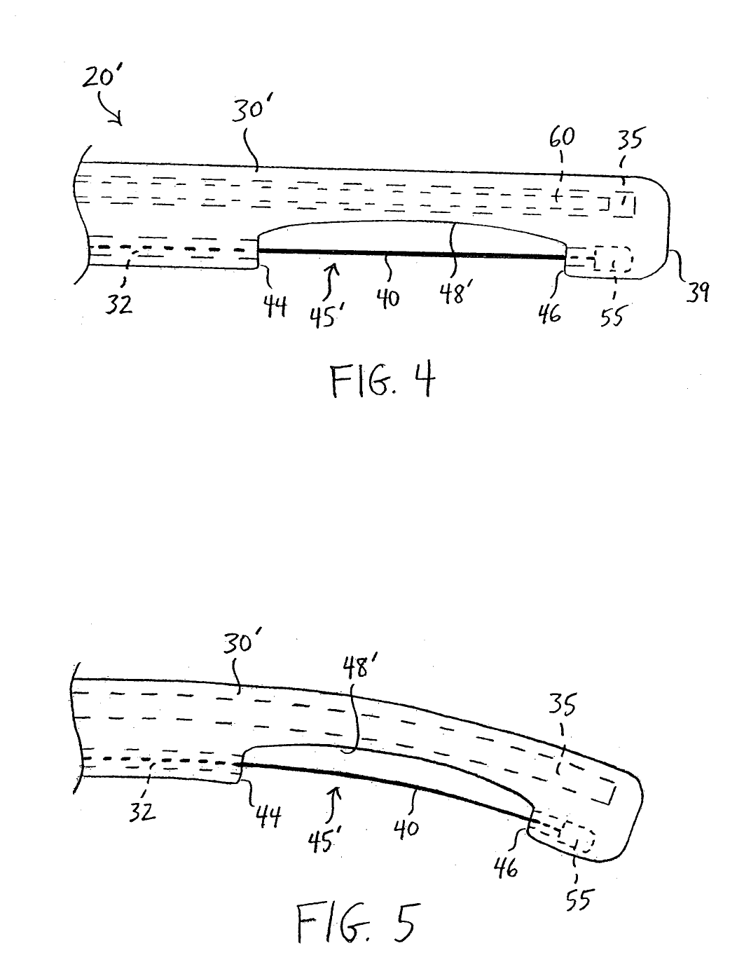

Referring now to FIG. 4, an alternative embodiment is shown. Apparatus 20′ is similar to the apparatus 20 of FIGS. 1-2, with a main exception that an indented surface 48′ comprises a curvature extending between the first end 44 and the second end 46 of a notched section 45′. The curvature of the indented surface 48′ may comprise a concave shape, thereby varying the distance between the catheter shaft and the cutting wire 40 along the notched section 45′, as depicted in FIG. 4.

In FIG. 4, the stiffening member 60 has been distally advanced to provide support to the cutting wire 40. The stiffening member 60 substantially inhibits bending of the notched section 45′ when positioned at this distal location. In FIG. 5, the stiffening member 60 has been retracted proximal to the notched section 45′, which allows a physician to tension the cutting wire 40 to cause the notched section 45′ to bend. For example, the actuator 79 may be retracted proximally with respect to the thumb ring 75 to tension the proximal end of the cutting wire 40, which in turn urges the affixation point 55 in a proximal direction with respect to the main body of the catheter 30 to cause the notched section 45′ to bend, as shown in FIG. 5.

Referring now to FIGS. 6A-6B, exemplary endoscopes that may be used in conjunction with apparatus 20 or 20′ during a polyp removal procedure are described. In FIG. 6A, a side-viewing endoscope 150 comprises a conventional endoscope having proximal and distal ends. The distal end of the endoscope 150 may comprise optical elements 173 and 174, which employ fiber optic components for illuminating and capturing an image to the side of, or distal to, the endoscope. Further, the endoscope 150 preferably comprises a working channel 161, which is sized to accommodate the catheter 30 therein for purposes of longitudinally advancing the catheter to a target site. A guiding channel 165 may be formed near the distal surface of the endoscope 150 to cause components advanced through the working channel 161 to exit at a predetermined angle with respect to a longitudinal axis of the endoscope 150. It will be apparent that while one working channel 161 is shown, the endoscope 150 may comprise at least one more additional lumen or channel, such as an auxiliary lumen.

The working channel 161 of the endoscope 110 may have an inner diameter of about 3.0-7.0 mm, while the overall diameter of the endoscope 110 may be about 10-14 mm. As noted above, the first outer diameter D₁ of the catheter 30 may range from about 2.0 mm to about 6.0 mm, and more preferably about 4.0 mm. Accordingly, the catheter 30 may be sized to be advanced through the working channel 161 of the endoscope 110. Such dimensions are provided for exemplary reference purposes and are not intended to be limiting.

In FIG. 6B, an end-viewing endoscope 150′, which alternatively may be used in conjunction with apparatus 20 or 20′ during a polyp removal procedure, is shown. The endoscope 150′ is similar to the endoscope 150 above, with the main exception that optical elements 173′ and 174′ are disposed on the distal end surface of the endoscope 150′. Further, a working channel 161′ extends through the distal end surface of the endoscope 150′, as shown in FIG. 6B. The working channel 161′ also is sized to accommodate the catheter 30 therein for purposes of longitudinally advancing the catheter to a target site. As shown, one auxiliary lumen 162′ is provided, although greater or fewer lumens/channels may be employed.

Referring now to FIG. 7, a method suitable for surgical treatment of a polyp in a bodily cavity, such as a colon, is described. In a first step, the endoscope 150′ has been maneuvered into a patient's colon 188 having tissue folds 189. The polyp 190 has a base portion 192 and a head portion 194 formed within the wall of the colon 188. The endoscope 150′ preferably is positioned such that its distalmost end is disposed just proximal to the polyp 190, as shown in FIG. 7.

Once the endoscope 150′ is positioned and the polyp 190 is imaged using the optical elements 173′ and 174′ of the endoscope, the catheter 30 may be distally advanced through the working lumen 161′ of the endoscope 150′ and positioned adjacent to the polyp 190, as shown in FIG. 7. The catheter 30 preferably is positioned such that the notched section 45, having the cutting wire 40 exposed, is in close proximity to the base portion 192 of the polyp 190, as shown in FIG. 7. At this time, the fiber optic components of the optical elements 173′ and 174′ of the endoscope 150′ may capture an image distal to the endoscope to ensure correct positioning of the catheter 30.

In a next step, an electrical current may be applied to the cutting wire 40 using the electrical source operably coupled to the connector 76 (see FIG. 3). At this time, a physician may manipulate the positioning of the catheter 30 to cut through the base portion 192 of the polyp 190. The provision of the stiffening member 60 at a location adjacent to the notched section 45 (see FIG. 1) may facilitate cutting of the polyp 190 by reinforcing the distal region of the catheter 30.

At any time during the procedure, fluid may be provided in the vicinity of the polyp 190, for example, for irrigation or cooling purposes. As noted above, the fluid may be injected via the injection port 77, through the first lumen 32, and may exit the catheter 30 in the vicinity of the notched section 45. Alternatively, such fluids may be provided through an auxiliary lumen of the catheter 30 and/or through the auxiliary lumen 162′ of the endoscope 150′. After the polyp 190 has been cut, a retrieval device, such as a forceps, may be used to capture and remove the polyp using known techniques.

If it becomes desirable to curve the distal region of the catheter 30, then the stiffening member 60 may be retracted proximal to the notched section 45. At this time, a proximal region of the cutting wire 40 may be tensioned, for example, by retracting the actuator 79 with respect to the thumb ring 75, as explained above with respect to FIG. 3. When the cutting wire 40 is tensioned, and the stiffening member 60 is removed or retracted, a distal portion of the catheter 30 including the notched section 45 may bend, as depicted in FIG. 5 above.

In accordance with one aspect, the apparatuses 20 and 20′ overcome at least some of the disadvantages associated with polyp removal using looped snares. In particular, the catheters 30 and 30′ may be used to remove relatively large or awkward polyps that may be difficult to excise using a looped snare.

Moreover, the apparatuses 20 and 20′ comprise advantages over sphincterotomes in which a cutting wire is bowed radially outward with respect to a lateral surface of the catheter. For example, the notched section 45 provides some protection from direct contact between the electrified cutting wire 40 and the body wall, e.g., the bowel wall.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

We claim:

1. Apparatus suitable for surgical treatment of a polyp, the apparatus comprising:
a catheter having proximal and distal regions, and further having a first lumen extending between the proximal and distal regions;
a cutting wire having proximal and distal ends, wherein the cutting wire extends through the first lumen of the catheter; and
at least one notched section having an indented surface formed in the distal region of the catheter, wherein the notched section is disposed in a lateral surface of the catheter at a location proximal to a distal tip of the catheter, and wherein at least a portion of the cutting wire is visibly exposed at the notched section,
wherein the exposed portion of the cutting wire is spaced apart from the indented surface.

2. The apparatus of claim 1 wherein the catheter comprises a first outer region disposed proximal to the notched section, wherein the exposed portion of the cutting wire does not extend substantially radially outward beyond the first outer region of the catheter.

3. The apparatus of claim 1 further comprising a stiffening member having proximal and distal ends, wherein the stiffening member is adapted to be distally advanced within a second lumen of the catheter to selectively provide support to the cutting wire.

4. The apparatus of claim 1 further comprising a stiffening member rigidly affixed adjacent to the notched section of the catheter to provide support to the cutting wire.

5. The apparatus of claim 1 wherein proximal retraction of the cutting wire is adapted to bend the distal region of the catheter.

6. The apparatus of claim 5 wherein the distal end of the cutting wire is affixed to an affixation member positioned near a distal end of the catheter.

7. The apparatus of claim 1 wherein the notched section of the catheter comprises first and second ends, and wherein the indented surface comprises a curvature formed between the first and second ends.

8. The apparatus of claim 7 wherein the curvature comprises a concave shape.

9. The apparatus of claim 1 wherein the notched section of the catheter comprises first and second ends, and a longitudinal length between the first and second ends is equal to or greater than a diameter of a base of a polyp.

10. A method suitable for surgical treatment of a polyp, the method comprising:
providing a catheter having proximal and distal regions, and further having a first lumen extending between the proximal and distal ends;
disposing a cutting wire having proximal and distal ends through the first lumen of the catheter, wherein at least a portion of the cutting wire is exposed in a notched section having an indented surface formed in the distal region of the catheter, wherein the exposed portion of the cutting wire is spaced apart from the indented surface; and
treating the polyp by engaging a portion of the polyp with the cutting wire.

11. The method of claim 10 wherein the catheter comprises a first outer region disposed proximal to the notched section, wherein the exposed portion of the cutting wire does not extend substantially radially outward beyond the first outer region of the catheter.

12. The method of claim 10 further comprising providing a stiffening member having proximal and distal ends, wherein the stiffening member is adapted to be distally advanced within a second lumen of the catheter to a position adjacent to the notched section to selectively provide support to the cutting wire.

13. The method of claim 10 further comprising providing a stiffening member rigidly affixed adjacent to the notched section of the catheter to provide support to the cutting wire.

14. The method of claim 10 further comprising proximally retracting the cutting wire to bend the distal region of the catheter.

15. The method of claim 10 further comprising distally advancing the catheter through a working lumen of an endoscope to treat the polyp.

16. Apparatus suitable for surgical treatment of a polyp, the apparatus comprising:
a catheter having proximal and distal regions, and further having a first and second lumens extending between the proximal and distal regions;
a cutting wire having proximal and distal ends, wherein the cutting wire extends through the first lumen of the catheter;
at least one notched section having an indented surface formed in the distal region of the catheter, wherein at least a portion of the cutting wire is visibly exposed at the notched section, and
a stiffening member having proximal and distal ends, wherein the stiffening member is adapted to be distally advanced within the second lumen of the catheter to selectively provide support to the cutting wire.

17. The apparatus of claim 16 wherein the exposed portion of the cutting wire is spaced apart from the indented surface.

18. The apparatus of claim 16 wherein the catheter comprises a first outer region disposed proximal to the notched section, wherein the exposed portion of the cutting wire does not extend substantially radially outward beyond the first outer region of the catheter.

19. The apparatus of claim 16 wherein proximal retraction of the cutting wire is adapted to bend the distal region of the catheter.

20. The apparatus of claim 16 wherein the notched section of the catheter comprises first and second ends, and wherein the indented surface comprises a concave curvature formed between the first and second ends.
